(19) United States
(12) Patent Application Publication
(10) Pub. No.: US 2026/0027275 A1
(43) Pub. Date: Jan. 29, 2026

(54) DIALYSIS KITS FOR PREPARING DIALYSIS FLUIDS AND RELATED SYSTEMS AND RELATED METHODS

(71) Applicant: Mozarc Medical US LLC, Minneapolis, MN (US)

(72) Inventors: Sukalyan Dutta, Minneapolis, MN (US); Augusto Piazza, Minneapolis, MN (US); Sarah J. Grimm, Minneapolis, MN (US); Samuel Johnson, Minneapolis, MN (US); Piero Bruschi, Minneapolis, MN (US)

(21) Appl. No.: 19/280,306

(22) Filed: Jul. 25, 2025

Related U.S. Application Data
(60) Provisional application No. 63/675,957, filed on Jul. 26, 2024.

(51) Int. Cl. A61M 1/28

(52) U.S. Cl. CPC A61M 1/287

(57) ABSTRACT

Home dialysis kits for preparing dialysis fluids are provided. The home dialysis kit comprises a first container comprising a first solution and a second container comprising a second solution. The first solution comprises at least one of a dextrose component, a pH agent, or any combination thereof. The second container comprises at least one of a calcium component, a magnesium component, a sodium component, a chloride component, a lactate component, or any combination thereof. The first solution and the second solution can be combined to produce a dialysis fluid. Related systems and related methods are also provided.

### CROSS-REFERENCE TO RELATED APPLICATIONS

This application claims priority to U.S. Provisional Patent Application No. 63/675,957, filed Jul. 26, 2024, and titled “DIALYSIS KITS FOR PREPARING DIALYSIS FLUIDS AND RELATED SYSTEMS AND RELATED METHODS,” the disclosure of which application is hereby incorporated herein by reference in its entirety.

### FIELD

The present disclosure relates to dialysis kits for preparing dialysis fluids and related systems and related methods, among other things.

### BACKGROUND

Peritoneal Dialysis (PD), including Automated Peritoneal Dialysis (APD) and Continuous Ambulatory Peritoneal Dialysis (CAPD), is a dialysis treatment. During PD, a catheter is placed in the peritoneal cavity and dialysate is introduced directly into the peritoneal cavity.

### SUMMARY

Some embodiments relate to a dialysis kit. In some embodiments, the home dialysis kit comprises a first container. In some embodiments, the first container comprises a first solution. In some embodiments, the first solution comprises a dextrose component. In some embodiments, the first solution comprises a pH agent. In some embodiments, the home dialysis kit comprises a second container. In some embodiments, the second container comprises a second solution. In some embodiments, the second solution has a concentration of at least 3 mM of a calcium ion. In some embodiments, the second solution has a concentration of at least 1 mM of a magnesium ion. In some embodiments, the second solution has a concentration of at least 250 mM of a sodium ion. In some embodiments, the second solution has a concentration of at least 200 mM of a chloride ion. In some embodiments, the second solution has a concentration of at least 80 mM of a lactate. In some embodiments, when the first container and the second container are connected to a home dialysis system, the first solution and the second solution are configured to be combined sufficient to produce a dialysis fluid having a concentration of 1% to 10% by weight of the dextrose component based on a total volume of the dialysis fluid.

In some embodiments, the first solution comprises 8.5% to 60% by weight of a dextrose component based on a total volume of the first solution.

In some embodiments, the pH agent is present in the first container in an amount sufficient for the first solution to have a pH of 0.1 to 3 when measured at a temperature of 20° C. to 30° C.

In some embodiments, the first solution comprises less than 1% by weight of a glucose degradation product based on a total weight of the first solution.

In some embodiments, the glucose degradation product comprises at least one of 5-hydroxymethyl furfural, 3-deoxyglucosone, glyoxal, methylglyoxal, acetaldehyde, or any combination thereof.

In some embodiments, the first solution comprises less than 0.04% by weight of 5-hydroxymethylfurfural (HMF) based on a total weight of the dextrose component.

In some embodiments, the second solution has a concentration of 10 mM to 30 mM of the calcium ion. In some embodiments, the second solution has a concentration of 2 mM to 6 mM of the magnesium ion. In some embodiments, the second solution has a concentration of 1000 mM to 3000 mM of the sodium ion. In some embodiments, the second solution has a concentration of 800 mM to 2200 mM of the chloride ion. In some embodiments, the second solution has a concentration of 350 mM to 1000 mM of the lactate.

In some embodiments, the second solution comprises less than 0.1 g of a solid precipitate.

In some embodiments, the first container comprises 0.1 L to 2 L of the first solution.

In some embodiments, the second container comprises 0.1 L to 2 L of the second solution.

Some embodiments relate to a method. In some embodiments, the method comprises obtaining a first container. In some embodiments, the first container comprises a first solution. In some embodiments, the first solution comprises a dextrose component. In some embodiments, the first solution comprises a pH agent. In some embodiments, the method comprises obtaining a second container. In some embodiments, the second container comprises a second solution. In some embodiments, the second solution has a concentration of at least 3 mM of a calcium ion. In some embodiments, the second solution has a concentration of at least 1 mM of a magnesium ion. In some embodiments, the second solution has a concentration of at least 250 mM of a sodium ion. In some embodiments, the second solution has a concentration of at least 200 mM of a chloride ion. In some embodiments, the second solution has a concentration of at least 80 mM of a lactate. In some embodiments, the method comprises connecting the first container and the second container to a home dialysis system. In some embodiments, the method comprises operating the home dialysis system to produce a dialysis fluid. In some embodiments, the method comprises supplying the dialysis fluid from the home dialysis system to a patient.

In some embodiments, the home dialysis system is configured to mix a portion of the first solution and a portion of the second solution, with water from a water source, to produce the dialysis fluid.

In some embodiments, the first solution comprises 8.5% to 60% by weight of a dextrose component based on a total volume of the first solution.

In some embodiments, the pH agent is present in the first container in an amount sufficient for the first solution to have a pH of 0.1 to 3 when measured at a temperature of 20° C. to 30° C.

In some embodiments, the first solution comprises less than 1% by weight of a glucose degradation product based on a total weight of the dextrose component.

In some embodiments, the glucose degradation product comprises at least one of 5-hydroxymethyl furfural, 3-deoxyglucosone, glyoxal, methylglyoxal, acetaldehyde, or any combination thereof.

In some embodiments, the first solution comprises less than 0.04% by weight of 5-hydroxymethylfurfural (HMF) based on a total weight of the dextrose component.

In some embodiments, the second solution has a concentration of 10 mM to 30 mM of the calcium ion. In some embodiments, the second solution has a concentration of 2 mM to 6 mM of the magnesium ion. In some embodiments, the second solution has a concentration of 1000 mM to 3000 mM of the sodium ion. In some embodiments, the second solution has a concentration of 800 mM to 2200 mM of the chloride ion. In some embodiments, the second solution has a concentration of 350 mM to 1000 mM of the lactate.

In some embodiments, the second solution comprises less than 0.1 g of a solid precipitate.

In some embodiments, at least one of the first container, the second container, or any combination thereof, has a volume of 0.1 L to 2 L.

### BRIEF DESCRIPTION OF DRAWINGS

FIG. 1 is a schematic diagram of a dialysis kit, according to some embodiments.

FIG. 2 is a flowchart of a method for using a dialysis kit, according to some embodiments.

FIG. 3 is a schematic diagram of a dialysis system, according to some embodiments.

FIG. 4 is a graphical view of concentration of 5-hydroxymethylfurfural (HMF) as a function of pH, according to some embodiments.

### DETAILED DESCRIPTION

Dialysis kits for preparing dialysis fluids are provided. The dialysis kits may be useful for preparing dialysis fluids at home, among other locations. The dialysis kits may comprise a first container comprising a first solution, and a second container comprising a second solution. The first solution and the second solution may independently comprise concentrated formulations useful for preparing dialysis fluids, such as, for example and without limitation, peritoneal dialysis fluids. In some embodiments, the first solution and the second solution may be combined and/or diluted with a water solution (e.g., purified tap water, etc.) to prepare a dialysis fluid. Each of the first solution and the second solution are independently formulated to provide dialysis kits having improved shelf life and reduced levels of glucose degradation products, while minimizing formation of solid precipitate(s). By providing concentrated formulations, the dialysis kits obviate the need for patients and/or medical professionals to handle large volume dialysis fluids (e.g., 3 L to 6 L bags).

The dialysis kits also enable on-demand production of dialysis fluids, with a high degree of tunability with respect to dextrose concentration, among many other parameters. For example, in some embodiments, a dextrose component is separated from electrolytes (e.g., at least one of a calcium component, a magnesium component, a sodium component, a chloride component, a lactate component, or any combination thereof). In some embodiments, by separating the dextrose component from the electrolytes, a concentration of the dextrose component can be adjusted in real-time, adjusted based on a patient's identified need, and/or adjusted during a therapy, among other things. In some embodiments, the pH can also be adjusted in this way (e.g., to a physiological pH). Related systems and related methods are also provided herein, among other things.

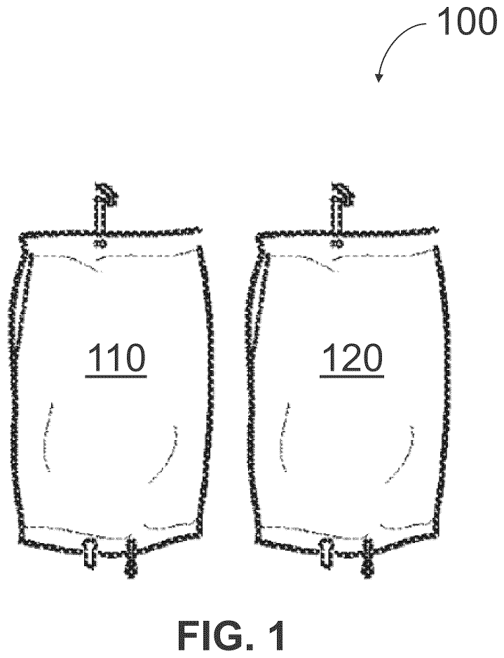

FIG. 1 is a schematic diagram of a dialysis kit 100, according to some embodiments. As shown in FIG. 1, in some embodiments, the dialysis kit 100 comprises a first container 110 and a second container 120. Although the first container 110 and the second container 120 are shown, it will be appreciated that the dialysis kit 100 may be provided in other configurations and/or may comprise additional components, without departing from the scope of this disclosure. In some embodiments, the dialysis kit 100 comprises a home dialysis kit. In some embodiments, the dialysis kit 100 comprises only the first container 110. For example, in some embodiments, the dialysis kit 100 does not comprise the second container 120. In some embodiments, the dialysis kit 100 comprises only the second container 120. For example, in some embodiments, the dialysis kit 100 does not comprise the first container 110.

A first solution may be contained in the first container 110. The first container 110 may comprise 0.1 L to 2 L of the first solution. For example, in some embodiments, the first container 110 comprises 0.2 L to 2 L, 0.3 L to 2 L, 0.4 L to 2 L, 0.5 L to 2 L, 0.6 L to 2 L, 0.7 L to 2 L, 0.8 L to 2 L, 0.9 L to 2 L, 1 L to 2 L, 1.1 L to 2 L, 1.2 L to 2 L, 1.3 L to 2 L, 1.4 L to 2 L, 1.5 L to 2 L, 1.6 L to 2 L, 1.7 L to 2 L, 1.8 L to 2 L, or 1.9 L to 2 L of the first solution.

In some embodiments, the first solution comprises a dextrose component. Non-limiting examples of the dextrose component include, for example and without limitation, at least one of dextrose, dextrose monohydrate, glucose polymers, amino acids, small molecule crystal glycerin, sorbitol, fructose, macromolecule gel, cationic polymer, polypeptide, any derivative thereof, or any combination thereof.

In some embodiments, the first solution comprises a pH agent. The pH agent may comprise a compound or other substance for adjusting a pH of the first solution to a pH range of 7 or less, such as, for example, 0.1 to 3. Non-limiting examples of the pH agent include, for example and without limitation, at least one of a hydrochloric acid, a sulfuric acid, a phosphoric acid, an acetic acid, an acetoacetic acid, a citric acid, an aluminum chloride, a zinc chloride, a copper chloride, a copper sulfate, an iron trichloride, or any combination thereof.

The first solution may comprise 8.5% to 60% by weight of the dextrose component based on a total volume of the first solution, or any range or subrange between 8.5% and 60%. For example, in some embodiments, the first solution comprises 8.5% to 60%, 8.5% to 55%, 8.5% to 50%, 8.5% to 45%, 8.5% to 40%, 8.5% to 35%, 8.5% to 30%, 8.5% to 25%, 8.5% to 20%, 8.5% to 15%, 8.5% to 10%, 10% to 60%, 15% to 60%, 20% to 60%, 25% to 60%, 30% to 60%, 35% to 60%, 40% to 60%, 45% to 60%, 50% to 60%, or 55% to 60%, by weight of the dextrose component based on the total volume of the first solution.

The first solution may comprise 1% to 10% by weight of the pH agent based on the total weight of the first solution, or any range or subrange between 1% and 10%. For example, in some embodiments, the first solution comprises 1% to 9%, 1% to 8%, 1% to 7%, 1% to 6%, 1% to 5%, 1% to 4%, 1% to 3%, 1% to 2%, 2% to 10%, 3% to 10%, 4% to 10%, 5% to 10%, 6% to 10%, 7% to 10%, 8% to 10%, or 9% to 10%, by weight of the pH agent based on the total weight of the first solution.

In some embodiments, the pH agent is present in an amount sufficient for the first solution to have a pH of 0.1 to 3 when measured at a temperature of 20° C. to 30° C. For example, in some embodiments, the pH agent is present in an amount sufficient for the first solution to have a pH of 0.1 to 3, 0.1 to 2, 0.1 to 1, 0.5 to 3, 1 to 3, 2 to 3, or any range or subrange between 0.1 and 3, when measured at a temperature of 20° C. to 30° C.

The first solution may have a pH of 0.1 to 3 when measured at a temperature of 20° C. to 30° C. For example, in some embodiments, the first solution has a pH of 0.1 to 3, 0.1 to 2, 0.1 to 1, 0.5 to 3, 1 to 3, 2 to 3, or any range or subrange between 0.1 and 3, when measured at a temperature of 20° C. to 30° C.

In some embodiments, the pH of the first solution is maintained at a constant value.

The first solution may comprise less than 1% by weight of a glucose degradation product based on a total weight of the dextrose component. For example, in some embodiments, the first solution comprises 0.001% to 1%, 0.001% to 0.9%, 0.001% to 0.8%, 0.001% to 0.7%, 0.001% to 0.6%, 0.001% to 0.5%, 0.001% to 0.4%, 0.001% to 0.3%, 0.001% to 0.2%, 0.001% to 0.1%, 0.001% to 0.01%, 0.01% to 1%, 0.1% to 1%, 0.2% to 1%, 0.3% to 1%, 0.4% to 1%, 0.5% to 1%, 0.6% to 1%, 0.7% to 1%, 0.8% to 1%, or 0.9% to 1% by weight of the glucose degradation product based on the total weight of the dextrose component.

The glucose degradation product comprises at least one of 5-hydroxymethyl furfural, 3-deoxyglucosone, glyoxal, methylglyoxal, acetaldehyde, or any combination thereof. It will be appreciated that the first solution may comprise low levels of other types of glucose degradation products, without departing from the scope of this disclosure.

The first solution may comprise less than 0.04% by weight of 5-hydroxymethylfurfural (HMF) based on a total weight of the dextrose component. For example, in some embodiments, the first solution comprises 0.001% to 0.04%, 0.001% to 0.03%, 0.001% to 0.02%, 0.001% to 0.01%, 0.01% to 0.04%, 0.02% to 0.04%, or 0.03% to 0.04% by weight of 5-hydroxymethylfurfural (HMF) based on the total weight of the dextrose component.

The first solution may be present in the first container in a liquid phase. For example, in some embodiments, the first solution does not comprise any solid, such as, for example and without limitation, in a form of a particle and/or a powder. In some embodiments, the first solution does not comprise a solid precipitate. In some embodiments, the first solution does not comprise a buffer solution. In some embodiments, the first solution does not comprise a buffer agent. In some embodiments, the first solution does not comprise at least one of a sodium bicarbonate, a sodium hydrogen bicarbonate, a sodium citrate, a sodium chloride, a sodium lactate, a sodium carbonate, a sodium acetate, or any combination thereof. In some embodiments, the first solution does not comprise any electrolytes.

A second solution may be contained in the second container 120. The second container 120 may comprise 0.1 L to 2 L of the second solution. For example, in some embodiments, the second container 120 comprises 0.2 L to 2 L, 0.3 L to 2 L, 0.4 L to 2 L, 0.5 L to 2 L, 0.6 L to 2 L, 0.7 L to 2 L, 0.8 L to 2 L, 0.9 L to 2 L, 1 L to 2 L, 1.1 L to 2 L, 1.2 L to 2 L, 1.3 L to 2 L, 1.4 L to 2 L, 1.5 L to 2 L, 1.6 L to 2 L, 1.7 L to 2 L, 1.8 L to 2 L, or 1.9 L to 2 L of the second solution.

The second solution may comprise an ion component (e.g., electrolytes). For example, in some embodiments, the second solution comprises ions, or compounds that dissolve and/or solubilize into ions. Non-limiting examples of the ion component include, for example and without limitation, at least one of a calcium component, a magnesium component, a sodium component, a chloride component, a lactate component, or any combination thereof.

In some embodiments, the second solution comprises the calcium component. In some embodiments, the calcium component comprises a calcium ion. In some embodiments, the calcium ion comprises Ca²⁺. In some embodiments, the calcium component comprises a calcium salt compound. In some embodiments, the calcium salt compound comprises at least one of a calcium salt, a hydrated calcium salt, or any combination thereof. Non-limiting examples of the calcium salt compound include, for example and without limitation, at least one of a calcium chloride, a calcium chloride dihydrate, or any combination thereof.

In some embodiments, the second solution comprises the magnesium component. In some embodiments, the magnesium component comprises a magnesium ion. In some embodiments, the magnesium ion comprises Mg²⁺. In some embodiments, the magnesium component comprises a magnesium salt compound. In some embodiments, the magnesium salt compound comprises at least one of a magnesium salt, a hydrated magnesium salt, or any combination thereof. Non-limiting examples of the magnesium salt compound include, for example and without limitation, at least one of a magnesium chloride, a magnesium chloride hexahydrate, or any combination thereof.

In some embodiments, the second solution comprises the sodium component. In some embodiments, the sodium component comprises a sodium ion. In some embodiments, the sodium ion comprises Nat. In some embodiments, the sodium component comprises a sodium salt compound. In some embodiments, the sodium salt compound comprises at least one of a sodium salt, a hydrated sodium salt, or any combination thereof. A non-limiting example of the sodium salt compound includes, for example and without limitation, sodium chloride, among others.

In some embodiments, the second solution comprises the chlorine component. In some embodiments, the chlorine component comprises a chloride ion. In some embodiments, the chlorine component comprises a chlorine salt compound. In some embodiments, a source of the chlorine component comprises at least one of the calcium component, the magnesium component, the sodium component, or any combination thereof.

In some embodiments, the second solution comprises the lactate component. In some embodiments, the lactate component comprises a lactate ion. In some embodiments, the lactate component comprises a lactate compound. A non-limiting example of the lactate component includes, for example and without limitation, sodium lactate, among others.

It will be appreciated that the second solution may comprise a component other than the lactate component, such as, for example and without limitation, at least one of a bicarbonate component, a citrate component, a carbonate component, an acetate component, or any combination thereof.

The second solution may have a pH that is different from a pH of the first solution. In some embodiments, the second solution has a pH of 4 to 8 when measured at a temperature of 20° C. to 30° C. For example, in some embodiments, the second solution has a pH of 5 to 8, 6 to 8, 7 to 8, 4 to 7, 4 to 6, 4 to 5, or any range or subrange between 4 and 8, when measured at a temperature of 20° C. to 30° C.

It will be appreciated that the second solution may comprise additional components without departing from the scope of this disclosure.

In some embodiments, the second solution and the first solution are different. For example, in some embodiments, the second solution does not comprise a dextrose component. In some embodiments, the second solution does not comprise a pH agent. In some embodiments, the first solution does not comprise the ion component. In some embodiments, the first solution does not comprise electrolytes. For example, in some embodiments, the first solution does not comprise at least one of the calcium component, the magnesium component, the sodium component, the lactate component, or any combination thereof. In some embodiments, the first solution does not comprise a buffer solution. In some embodiments, the first solution does not comprise a buffer agent. In some embodiments, the first solution does not comprise at least one of a bicarbonate, a sodium bicarbonate, a sodium hydrogen bicarbonate, a sodium citrate, a sodium chloride, a sodium lactate, a sodium carbonate, a sodium acetate, or any combination thereof.

The second solution may be formulated to achieve a concentration of calcium ions in the second solution. For example, in some embodiments, the second solution has a concentration of at least 3 mM of a calcium ion. In some embodiments, the second solution has a concentration of 10 mM to 30 mM, 10 mM to 28 mM, 10 mM to 26 mM, 10 mM to 25 mM, 10 mM to 24 mM, 10 mM to 22 mM, 10 mM to 20 mM, 10 mM to 18 mM, 10 mM to 16 mM, 10 mM to 15 mM, 10 mM to 14 mM, 10 mM to 12 mM, 12 mM to 30 mM, 14 mM to 30 mM, 15 mM to 30 mM, 16 mM to 30 mM, 18 mM to 30 mM, 20 mM to 30 mM, 22 mM to 30 mM, 24 mM to 30 mM, 25 mM to 30 mM, 26 mM to 30 mM, or 28 mM to 30 mM of the calcium ion, or any range or subrange between 3 mM and 30 mM.

The second solution may be formulated to achieve a concentration of magnesium ions in the second solution. For example, in some embodiments, the second solution has a concentration of at least 1 mM of a magnesium ion. In some embodiments, the second solution has a concentration of 2 mM to 6 mM, 2 mM to 5 mM, 2 mM to 4 mM, 2 mM to 3 mM, 3 mM to 6 mM, 4 mM to 6 mM, or 5 mM to 6 mM of the magnesium ion, or any range or subrange between 1 mM and 6 mM.

The second solution may be formulated to achieve a concentration of sodium ions in the second solution. For example, in some embodiments, the second solution has a concentration of at least 250 mM of a sodium ion. In some embodiments, the second solution has a concentration of 250 mM to 3000 mM, 250 mM to 2750 mM, 250 mM to 2500 mM, 250 mM to 2250 mM, 250 mM to 2000 mM, 250 mM to 1750 mM, 250 mM to 1500 mM, 250 mM to 1500 mM, 250 mM to 1250 mM, 250 mM to 1000 mM, 250 mM to 750 mM, 250 mM to 500 mM, 500 mM to 3000 mM, 750 mM to 3000 mM, 1000 mM to 3000 mM, 1250 mM to 3000 mM, 1500 mM to 3000 mM, 1750 mM to 3000 mM, 2000 mM to 3000 mM, 2250 mM to 3000 mM, 2500 mM to 3000 mM, or 2750 mM to 3000 mM of the sodium ion, or any range or subrange between 250 mM and 3000 mM.

The second solution may be formulated to achieve a concentration of chloride ions in the second solution. For example, in some embodiments, the second solution has a concentration of at least 200 mM of a chloride ion. In some embodiments, the second solution has a concentration of 200 mM to 2200 mM, 200 mM to 2000 mM, 200 mM to 1800 mM, 200 mM to 1600 mM, 200 mM to 1500 mM, 200 mM to 1400 mM, 200 mM to 1200 mM, 200 mM to 1000 mM, 200 mM to 800 mM, 200 mM to 600 mM, 200 mM to 500 mM, 200 mM to 400 mM, 400 mM to 2200 mM, 500 mM to 2200 mM, 600 mM to 2200 mM, 800 mM to 2200 mM, 1000 mM to 2200 mM, 1200 mM to 2200 mM, 1400 mM to 2200 mM, 1500 mM to 2200 mM, 1600 mM to 2200 mM, 1800 mM to 2200 mM, or 2000 mM to 2200 mM of the chloride ion, or any range or subrange between 200 mM and 2200 mM.

The second solution may be formulated to achieve a concentration of lactate in the second solution. For example, in some embodiments, the second solution has a concentration of at least 80 mM of a lactate. In some embodiments, the second solution has a concentration of 80 mM to 1000 mM, 80 mM to 950 mM, 80 mM to 900 mM, 80 mM to 850 mM, 80 mM to 800 mM, 80 mM to 750 mM, 80 mM to 700 mM, 80 mM to 650 mM, 80 mM to 600 mM, 80 mM to 550 mM, 80 mM to 500 mM, 80 mM to 450 mM, 80 mM to 400 mM, 80 mM to 350 mM, 80 mM to 300 mM, 80 mM to 250 mM, 80 mM to 200 mM, 80 mM to 150 mM, 100 mM to 1000 mM, 150 mM to 1000 mM, 200 mM to 1000 mM, 250 mM to 1000 mM, 300 mM to 1000 mM, 350 mM to 1000 mM, 400 mM to 1000 mM, 450 mM to 1000 mM, 500 mM to 1000 mM, 550 mM to 1000 mM, 600 mM to 1000 mM, 650 mM to 1000 mM, 700 mM to 1000 mM, 750 mM to 1000 mM, 800 mM to 1000 mM, 850 mM to 1000 mM, 900 mM to 1000 mM, or 950 mM to 1000 mM of the lactate, or any range or subrange between 80 mM and 1000 mM.

The second solution may be present in the second container in a liquid phase. For example, in some embodiments, the second solution does not comprise any solid, such as, for example and without limitation, in a form of a particle and/or a powder. In some embodiments, the second solution does not comprise a solid precipitate. The second solution may comprise less than 0.1 g of a solid precipitate. For example, in some embodiments, the second solution may comprise 0.001 g to 0.1 g, 0.001 g to 0.01 g, or 0.01 g to 0.1 g of a solid precipitate.

In some embodiments, when the first container and the second container are stored at a temperature of 5° C. to 40° C., each of the first solution and the second solution has a shelf life of 1 month to 36 months, or any range or subrange between 1 month and 36 months and/or 5° C. and 40° C. In some embodiments, each of the first solution and the second solution has a shelf life of In some embodiments, a shelf life is a duration over which the first solution forms less than 1% by weight of a glucose degradation product(s) and/or less than 0.04% by weight of 5-HMF, based on the total weight the dextrose component. In some embodiments, the shelf life is (or is also) a duration over which the second solution forms less than 0.1 g of a solid precipitate.

In some embodiments, the first solution comprises 0.001% to 1%, 0.001% to 0.9%, 0.001% to 0.8%, 0.001% to 0.7%, 0.001% to 0.6%, 0.001% to 0.5%, 0.001% to 0.4%, 0.001% to 0.3%, 0.001% to 0.2%, 0.001% to 0.1%, 0.001% to 0.01%, 0.01% to 1%, 0.1% to 1%, 0.2% to 1%, 0.3% to 1%, 0.4% to 1%, 0.5% to 1%, 0.6% to 1%, 0.7% to 1%, 0.8% to 1%, or 0.9% to 1% by weight of the glucose degradation product and/or the solid precipitate based on the total weight of the dextrose component.

In some embodiments, the second solution comprises less than 1 g, less than 0.1 g, or less than 0.01 g of the solid precipitate, or any range or subrange between undetectable levels of the solid precipitate and 1 g.

In some embodiments, the shelf life of the first solution is 1 month to 36 months, 1 month to 34 months, 1 month to 32 months, 1 month to 30 months, 1 month to 28 months, 1 month to 26 months, 1 month to 24 months, 1 month to 22 months, 1 month to 20 months, 1 month to 18 months, 1 month to 16 months, 1 month to 14 months, 1 month to 12 months, 1 month to 10 months, 1 month to 8 months, 1 month to 6 months, 1 month to 4 months, 1 month to 2 months, 2 months to 36 months, 4 months to 36 months, 6 months to 36 months, 8 months to 36 months, 10 months to 36 months, 12 months to 36 months, 14 months to 36 months, 16 months to 36 months, 18 months to 36 months, 20 months to 36 months, 22 months to 36 months, 24 months to 36 months, 26 months to 36 months, 28 months to 36 months, 30 months to 36 months, 32 months to 36 months, 34 months to 36 months, or any range or subrange between 1 month and 36 months. In some embodiments, the shelf life of the first solution is 3 years. In some embodiments, the shelf life of the first solution is 2 years.

In some embodiments, the shelf life of the second solution is 1 month to 36 months, 1 month to 34 months, 1 month to 32 months, 1 month to 30 months, 1 month to 28 months, 1 month to 26 months, 1 month to 24 months, 1 month to 22 months, 1 month to 20 months, 1 month to 18 months, 1 month to 16 months, 1 month to 14 months, 1 month to 12 months, 1 month to 10 months, 1 month to 8 months, 1 month to 6 months, 1 month to 4 months, 1 month to 2 months, 2 months to 36 months, 4 months to 36 months, 6 months to 36 months, 8 months to 36 months, 10 months to 36 months, 12 months to 36 months, 14 months to 36 months, 16 months to 36 months, 18 months to 36 months, 20 months to 36 months, 22 months to 36 months, 24 months to 36 months, 26 months to 36 months, 28 months to 36 months, 30 months to 36 months, 32 months to 36 months, 34 months to 36 months, or any range or subrange between 1 month and 36 months. In some embodiments, the shelf life of the second solution is 3 years. In some embodiments, the shelf life of the second solution is 2 years.

In some embodiments, the shelf life of the first solution and the second solution is the same. In some embodiments, the shelf life of the first solution and the second solution is different.

In some embodiments, when the first container and the second container are connected to a home dialysis system, the first solution and the second solution are combined sufficient to produce a dialysis fluid having a concentration of 1% to 10% by weight of the dextrose component based on a total volume of the dialysis fluid. In some embodiments, the first solution and the second solution are combined sufficient to produce a dialysis fluid having a concentration of 1% to 10%, 1% to 9%, 1% to 8%, 1% to 7%, 1% to 6%, 1% to 5%, 1% to 4%, 1% to 3%, 1% to 2%, 2% to 10%, 3% to 10%, 4% to 10%, 5% to 10%, 6% to 10%, 7% to 10%, 8% to 10%, 9% to 10%, or any range or subrange between 1% and 10%, by weight of the dextrose component based on the total volume of the dialysis fluid. In some embodiments, the dialysis fluid is a peritoneal dialysis fluid. In some embodiments, the dialysis fluid does not have a concentration of 1.5% by weight of the dextrose component based on the total volume of the dialysis fluid. In some embodiments, the dialysis fluid does not have a concentration of 2.5% by weight of the dextrose component based on the total volume of the dialysis fluid. In some embodiments, the dialysis fluid does not have a concentration of 4.25% by weight of the dextrose component based on the total volume of the dialysis fluid.

In some embodiments, at least one of the first solution, the second solution, or any combination thereof, is diluted with a water solution sufficient to produce the dialysis fluid. For example, in some embodiments, the water solution comprises at least one of a purified water, a deionized water, a reverse osmosis water, a water for injection, a sterilized water, or any combination thereof. The first solution may be diluted 2 to 40 times, or any range or subrange between 2 and 40. For example, in some embodiments, the first solution is diluted 3 to 40 times, 4 to 40 times, 5 to 40 times, 10 to 40 times, 15 to 40 times, 20 to 40 times, 25 to 40 times, 30 to 40 times, or 35 to 40 times. The second solution may be diluted 2 to 40 times, or any range or subrange between 2 and 40. For example, in some embodiments, the second solution is diluted 3 to 40 times, 4 to 40 times, 5 to 40 times, 10 to 40 times, 15 to 40 times, 20 to 40 times, 25 to 40 times, 30 to 40 times, or 35 to 40 times.

In some embodiments, at least one of the first solution, the second solution, or any combination thereof, is combined with the water solution sufficient to produce the dialysis fluid in a volume of 10 L to 25 L, or any range or subrange between 10 L and 25 L. For example, in some embodiments, the dialysis fluid has a volume of 10 L to 24 L, 10 L to 22 L, 10 L, to 20 L, 10 L to 18 L, 10 L to 16 L, 10 L to 15 L, 10 L to 14 L, 10 L to 12 L, 12 L to 25 L, 14 L to 25 L, 15 L to 25 L, 16 L to 25 L, 18 L to 25 L, 20 L to 25 L, 22 L to 25 L, or 24 L to 25 L.

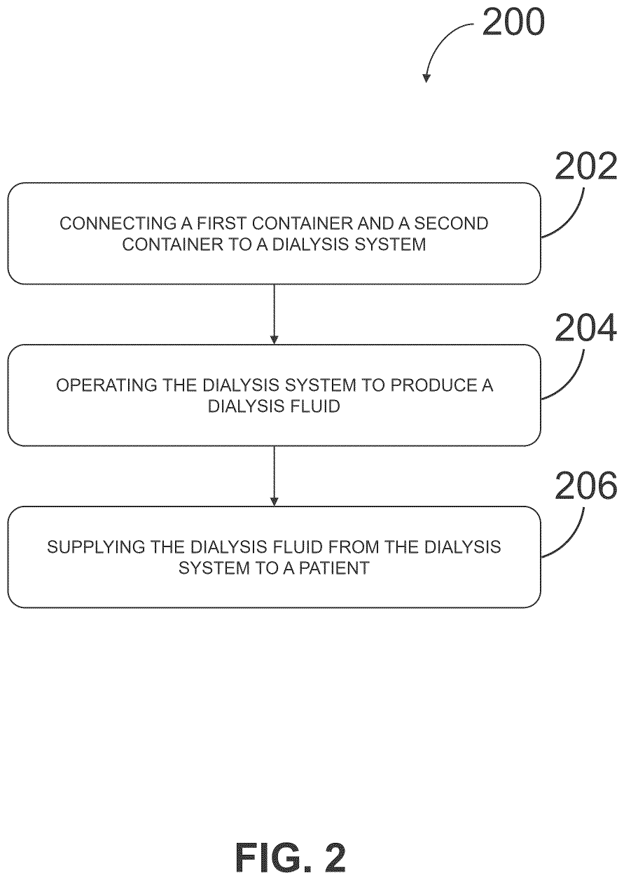

FIG. 2 is a flowchart of a method 200 for using a dialysis kit, according to some embodiments. As shown in FIG. 2, in some embodiments, the method 200 may comprise connecting 202 a first container and a second container to a dialysis system; operating 204 the dialysis system to produce a dialysis fluid; supplying 206 the dialysis fluid from the dialysis system to the patient. In some embodiments, the method 200 further comprises adjusting an amount of the first solution and/or the second solution to obtain a second dialysis fluid, wherein the second dialysis fluid has different concentration than the dialysis fluid. The method 200 may then repeat step 204.

At step 202, the method 200 may comprise connecting 202 a first container and a second container to a dialysis system. In some embodiments, the connecting 202 comprises fluidly coupling the first container and the second container to the dialysis system. For brevity, the first container and the second container are not repeated here. However, it will be appreciated that the first container and/or the second container may comprise any one or more of the first containers and second containers disclosed herein. The dialysis system may comprise a home dialysis system. For example, in some embodiments, the dialysis system comprises a home peritoneal dialysis system. Other types of dialysis systems, whether at a medical facility or at a home, may be employed such as, for example and without limitation, at least one of a hemodialysis system, a hemodiafiltration system, a hemofiltration system, or any combination thereof.

At step 204, the method 200 may comprise operating 204 the dialysis system to produce a dialysis fluid. In some embodiments, the operating 204 comprises mixing the first solution from the first container, with the second solution from the second container. In some embodiments, the operating 204 comprises diluting the first solution from the first container with a water solution. In some embodiments, the operating 204 comprises diluting the second solution from the second container with a water solution. In some embodiments, the operating 204 comprises mixing the first solution from the first container and the second solution from the second container to obtain a mixture, and diluting the mixture with a water solution. In some embodiments, the operating 204 comprising mixing and diluting a sufficient amount of the first solution and a sufficient amount of the second solution, with a water solution, so as to form a dialysis fluid suitable for a dialysis treatment, such as, for example and without limitation, a peritoneal dialysis treatment.

At step 206, the method 200 may comprise supplying 206 the dialysis fluid from the dialysis system to the patient. In some embodiments, the supplying 206 comprises flowing the dialysis fluid at a temperature and at a flow rate suitable for delivering a dialysis treatment, such as, for example and without limitation, a peritoneal dialysis treatment, to the patient.

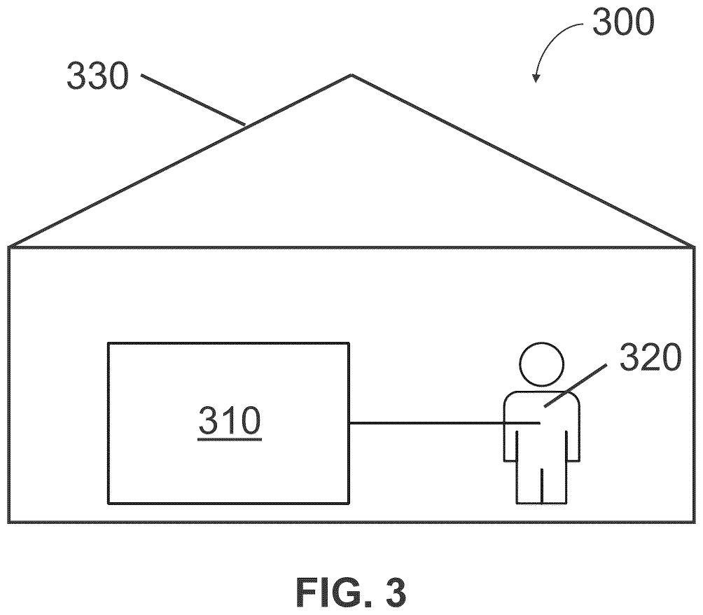

FIG. 3 is a schematic diagram of a home dialysis system 300, according to some embodiments. As shown in FIG. 3, in some embodiments, the home dialysis system 300 comprises a dwelling 330. The dwelling 330 comprises a dialysis system 310, such as, for example and without limitation, a peritoneal dialysis system, among other dialysis systems, and a patient 320. The dialysis system 310 delivers a dialysis treatment to the patient 320.

### Example 1

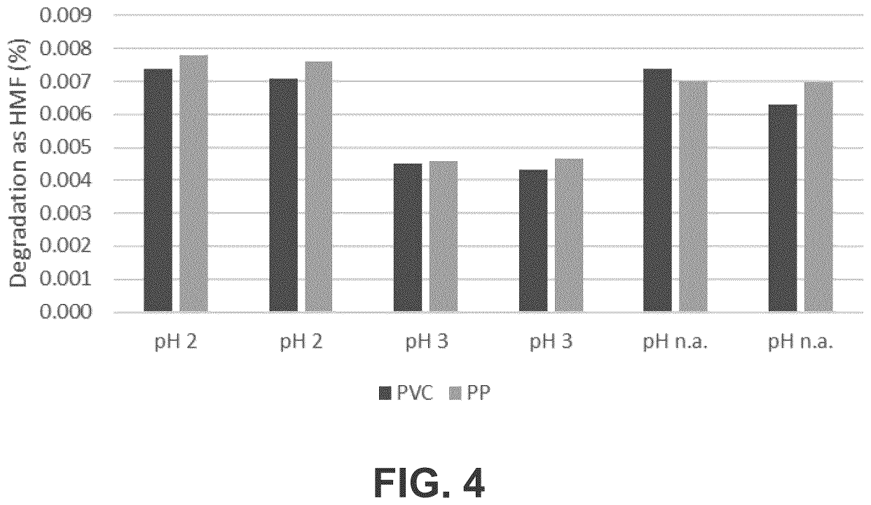

A pH of the first solution was adjusted to minimize reactions involving dextrose to glucose degradation products (GDPs). To evaluate pH level on the formation of glucose degradation products, the first solution was put into two different containers and a pH of the first solution in each container was adjusted to a pH of 2, 3, or the pH was not adjusted (n.a.). One of the containers was made of polyvinyl chloride (PVC). The other container was made of polypropylene. The containers comprising the first solution underwent a sterilization process and then were placed in an acceleration chamber for one (1) month at 40° C. and 75% relative humidity. After 1 month, the first solutions in each of the containers was analyzed for 5-hydroxymethylfurfural (HMF). The results are summarized in FIG. 4. FIG. 4 is a graphical view of concentration of 5-hydroxymethylfurfural (HMF) as a function of pH, according to some embodiments. As shown in FIG. 4, the lowest amount of HMF was observed for pH level of 3.

### Example 2

A second solution was prepared with varying concentrations of lactate to evaluate the extent to which solid precipitates are formed. The concentration of electrolytes and lactate were studied at varying concentrations. It was observed that a second solution 20 times more concentrated than a peritoneal dialysis fluid can be prepared without forming any solid precipitates.

What is claimed is:

1. A home dialysis kit comprising:
a first container comprising a first solution,
wherein the first solution comprises:
a first dextrose component; and
a pH agent; and
a second container comprising a second solution,
wherein the second solution comprises:
at least 3 mM of a calcium component;
at least 1 mM of a magnesium component;
at least 250 mM of a sodium component;
at least 200 mM of a chloride component; and
at least 80 mM of a lactate component;
wherein the second solution does not comprise a second dextrose component;
wherein, when the first container and the second container are connected to a home dialysis system, the first solution and the second solution are configured to be combined sufficient to produce a dialysis fluid having a concentration of 1% to 10% by weight of the first dextrose component based on a total volume of the dialysis fluid.

2. The home dialysis kit of claim 1, wherein the first solution comprises:
8.5% to 60% by weight of a dextrose component based on a total volume of the first solution.

3. The home dialysis kit of claim 1, wherein the pH agent is present in the first container in an amount sufficient for the first solution to have a pH of 0.1 to 3 when measured at a temperature of 20° C. to 30° C.

4. The home dialysis kit of claim 3, wherein the first solution comprises less than 1% by weight of a glucose degradation product based on a total weight of the first dextrose component.

5. The home dialysis kit of claim 4, wherein the glucose degradation product comprises at least one of 5-hydroxymethyl furfural, 3-deoxyglucosone, glyoxal, methylglyoxal, acetaldehyde, or any combination thereof.

6. The home dialysis kit of claim 1, wherein the first solution comprises less than 0.04% by weight of 5-hydroxymethylfurfural (HMF) based on a total weight of the first dextrose component.

7. The home dialysis kit of claim 1, wherein the second solution comprises:
10 mM to 30 mM of the calcium component;
2 mM to 6 mM of the magnesium component;
1000 mM to 3000 mM of the sodium component;
800 mM to 2200 mM of the chloride component; and
350 mM to 1000 mM of the lactate component.

8. The home dialysis kit of claim 1, wherein the second solution comprises less than 0.1 g of a solid precipitate.

9. The home dialysis kit of claim 1, wherein the first container comprises 0.1 L to 2 L of the first solution.

10. The home dialysis kit of claim 1, wherein the second container comprises 0.1 L to 2 L of the second solution.

11. A method comprising:
connecting a first container and a second container to a home dialysis system,
wherein the first container comprises a first solution,
wherein the first solution comprises:
a dextrose component; and
a pH agent;
wherein the second container comprises a second solution,
wherein the second solution comprises:
at least 3 mM of a calcium component;
at least 1 mM of a magnesium component;
at least 250 mM of a sodium component;
at least 200 mM of a chloride component; and
at least 80 mM of a lactate component;
operating the home dialysis system to produce a dialysis fluid; and
supplying the dialysis fluid from the home dialysis system to a patient.

12. The method of claim 11, wherein the home dialysis system is configured to mix a sufficient amount of the first solution and a sufficient amount of the second solution, with a sufficient amount of a water solution, to produce the dialysis fluid.

13. The method of claim 11, wherein the first solution comprises:
8.5% to 60% by weight of a dextrose component based on a total volume of the first solution.

14. The method of claim 11, wherein the pH agent is present in the first container in an amount sufficient for the first solution to have a pH of 0.1 to 3 when measured at a temperature of 20° C. to 30° C.

15. The method of claim 11, wherein the first solution comprises less than 1% by weight of a glucose degradation product based on a total weight of the dextrose component.

16. The method of claim 15, wherein the glucose degradation product comprises at least one of 5-hydroxymethyl furfural, 3-deoxyglucosone, glyoxal, methylglyoxal, acetaldehyde, or any combination thereof.

17. The method of claim 11, wherein the first solution comprises less than 0.04% by weight of 5-hydroxymethylfurfural (HMF) based on a total weight of the dextrose component.

18. The method of claim 11, wherein the second solution comprises:
10 mM to 30 mM of the calcium component;
2 mM to 6 mM of the magnesium component;
1000 mM to 3000 mM of the sodium component;
800 mM to 2200 mM of the chloride component; and
350 mM to 1000 mM of the lactate component.

19. The method of claim 11, wherein the second solution comprises less than 0.1 g of a solid precipitate.

20. The method of claim 11, wherein at least one of the first container, the second container, or any combination thereof, has a volume of 0.1 L to 2 L.
